(19) United States
(12) Patent Application Publication
(10) Pub. No.: US 2011/0038947 A1
(43) Pub. Date: Feb. 17, 2011

(54) INORGANIC PHOSPHOR, OBTAINABLE BY WET MILLING

(75) Inventors: Marc Maurer, Village-Neuf (FR); Niklaus Raetzo, Reinach (CH); Rene Fischer, Biel-Benken (CH)

Correspondence Address: BASF Corporation;Patent Department, 500 White Plains Road, P.O. Box 2005, Tarrytown, NY, 10591, US

(73) Assignee: BASF SE, Tarrytown, NY (US)

(21) Appl. No.: 12/744,932

(22) PCT Filed: Dec. 5, 2008
(86) PCT No.: PCT/EP08/66843

§ 371 Date: Oct. 15, 2010

(30) Foreign Application Priority Data
Dec. 14, 2007 (EP) 07123206.0

(51) Int. Cl. C09K 11/77; C09K 11/08; C09K 11/56; C09K 11/68; A61K 8/19

(52) U.S. Cl. 424/600; 252/301.4 R; 252/301.4 P; 252/301.4 S; 252/301.6 R; 252/301.6 P; 252/301.5

(57) ABSTRACT

The invention relates to inorganic phosphors, obtainable by wet milling and having a particle size distribution of D90<5 μm, to a method for producing said pigments and the use thereof. The use of wet milled inorganic phosphor particles wherein 90 percent of the particles have a diameter of equal to or less than 5 μm, especially equal to or less than 3 μm, very especially equal to or less than 1 μm can provide for an improved fluorescence in contrast to the general believe.

The invention relates to inorganic phosphors, obtainable by wet milling and having a particle size distribution of D90≦5 μm, to a method for producing said pigments and the use thereof. The use of wet milled inorganic phosphor particles wherein 90 percent of the particles have a diameter of equal to or less than 5 μm, especially equal to or less than 3 μm, very especially equal to or less than 1 μm can provide for an improved fluorescence in contrast to the general believe.

U.S. Pat. No. 6,344,261B1 concerns a printed valuable document with at least one authentication feature in the form of a luminescent substance based on a host lattice doped with at least one rare earth metal. Preferably, the luminescent substance has a garnet structure which satisfies with the general formula A₃Cr₅₋ₓAlₓO₁₂, where A stands for an element selected from the group consisting of scandium, yttrium, the lanthanides and the actinides, and the index x fulfils the condition 0<x<4.99. Even more preferred are luminescent substances Y_{3-z}Nd_{z}Cr₅₋ₓAlₓO₁₂, Y_{3-z}Yb_{z}Cr₅₋ₓAlₓO₁₂ and Y_{3-z}Pr_{z}Cr₅₋ₓAlₓO₁₂, where the index z fulfils the condition 0<z<1. In Example 1 of U.S. Pat. No. 6,344,261B1 the production of Y_{2.75}Nd_{0.2}Yb_{0.2}Cr_{0.8}Cr_{0.8}Al_{4.2}O₁₂ is described. In order to achieve the finest grain size, the powder is milled in water with a stirring ball mill until an average grain size of less than 1 μm is produced.

EP-A-1842892 discloses a UV-emitting phosphor and lamp containing the same. The phosphor is a praseodymium-activated pyrophosphate-based phosphor which may be represented by the general formula (Ca₂₋ₓ, Srₓ)P₂O₇:Pr where 0≦x≦2.

According to Example 1 of EP-A-1842892 Ca₂P₂O₇:Pr may be prepared by thoroughly dry blending the appropriate reactants, then firing the blended materials in a reducing atmosphere, preferably for 2-4 hours at 1000° C.-1200° C. in a 5% H₂-95% N₂ atmosphere. The fired cakes may be softened by soaking for 2-12 hours in de-ionized water and then wet-sieved-60 mesh and dried. Alternatively, the dry fired cakes may be broken into smaller pieces, ground and then dry sifted-60 mesh. The phosphor powder can be wet-milled to the appropriate size using a ball-milling technique with a minimal loss in brightness due to particle damage.

WO2007042653 relates to rare earth borate which is embodied in the form of a liquid phase suspension of substantially monocrystalline particles whose mean size ranges from 100 to 400 nm. Said borate is produced according a method consisting in roasting a rare earth borocarbonate or hydroxyborocarbonate with a temperature which is sufficient for forming a borate and obtaining a product whose specific surface area is equal to or greater than 3 m²/g and in carrying out the humid grinding of the roasted product. The inventive borate can be used in the form of luminophore, in particular, for producing a luminescent transparent material. In Example 1 of WO2007042653 a wet milling step is used to deagglomerate the phosphor particles after calcination.

It is a general believe that the brightness of the fluorescence conversely declines to a remarkable degree when the particle size of the fluorescent substance particles becomes less than 3 μm, in particular less than 1 μm due to the application of conventional milling techniques. Reference is made, for example, to chapter 4.1.6.4 of “Inorganic Phosphors, Compositions, Preparations and Optical Properties” edited by CRC Press (Author: M. Yen; Marvin J. Weber), wherein it is stated that strong milling is lowering the luminescence efficiency due to defects created on the crystals.

In chapter 4.2.2.2 it is stated that grinding in ball mills damages the particles and causes a decrease in luminescence efficiency. In chapter 4.4.3 (Dispersion) it is mentioned that hard mechanical dispersions have to be avoided since luminescence properties are then degraded.

Surprisingly, the present inventors have shown that the use of luminescent, especially fluorescent pigment particles wherein 90 percent of the particles have a diameter of equal to or less than 5 μm, especially equal to or less than 3 μm, very especially equal to or less than 1 μm provide for an improved fluorescence in contrast to the general believe.

Accordingly, the present invention relates to inorganic phosphors, obtainable by wet milling, wherein the mill is operated at power densities >0.5 kW per litre of grinding space and the luminescence (fluorescence, or phosphorescence) intensity of the wet milled inorganic phosphor is at least about 50%, especially 70%, very especially 90% of the luminescence intensity of the inorganic phosphor, which is used as starting material in the milling process.

The powder batch of phosphor particles also has a narrow particle size distribution, such that the majority of particles are substantially the same size. Preferably, at least about 90 weight percent of the particles and more preferably at least about 95 weight percent of the particles are not larger than twice the average particle size. Thus, when the average particle size is about 2 μm, it is preferred that at least about 90 weight percent of the particles are not larger than 4 μm and it is more preferred that at least about 95 weight percent of the particles are not larger than 4 μm. As used herein, the average particle size is the volume average particle size.

Further, it is preferred that at least about 90 weight percent of the particles are not larger than about 1.5 times the average particle size. Thus, when the average particle size is about 2 μm, it is preferred that at least about 90 weight percent of the particles are not larger than about 3 μm.

In addition, the phosphor particles of the present invention are characterized by a distribution coefficient (D₁₀+D₉₀)/D₅₀<1.2, especially <1.0.

In a preferred embodiment of the present invention the average particle size and/or the D₅₀ of the inorganic phosphors is below 0.4 μm, especially below 0.2 μm.

The inorganic phosphor (crude inorganic phosphor), which is used as starting material in the milling process can be a non-milled inorganic phosphor obtained by a sinter-process.

The inorganic phosphors of the present invention may have a small particle size distribution and surprising chemical, mechanical and heat resistance combined with good photophysical properties, such as luminescence intensity (quantum yield).

The term “luminescence” means the emission of light in the visible, UV- and IR-range after input of energy. The luminescent material can be a fluorescent material, or a phosphorescent material. Such luminescent materials exhibit a characteristic emission of electromagnetic energy in response to an energy source generally without any substantial rise in temperature.

The milling is conducted “wet”, i.e. in liquid media. The general milling conditions can vary depending on the feed material, residence time, impeller speeds, and milling media particle size. Suitable conditions and residence times are described in the Examples. These conditions can be varied to obtain the desired size within the range of 0.01 to about 5 μm, especially 0.02 to about 1 μm.

The milling process of the present invention can, in principal, be performed in a neutral liquid media, such as, for example, organic polar solvents, such as alcohols etc., or in organic apolar solvents, such as dichloromethane, chlorobenzene, pentane, hexane, cyclohexane, toluene etc., or mixtures thereof, but is preferably performed in water and optionally a neutral, polar organic solvent.

Examples of the neutral, polar organic solvent are acetamide, formamide, methylacetamide, methylformamide, caprolactam, valerolactam, 1,1,2,2-tetramethylurea, dimethyl sulfoxide, sulfolane, nitromethane, nitrobenzene, acetonitrile, methanol, ethylene carbonate, dimethylacetamide, dimethylformamide and N-methylpyrrolidone, preferably dimethyl sulfoxide, dimethylformamide or N-methylpyrrolidone, especially N-methylpyrrolidone, and a mixture of a plurality of neutral liquids of same overall polarity.

Preferably, solvents are used, which do not absorb in the UV region.

If water and a neutral, polar liquid are used, the amount of neutral, polar liquid is from 1 to 30% by weight, preferably from 3 to 20% by weight, especially from 5 to 10% by weight, based on the total amount of liquid and water.

The milling equipment used to mill the parent inorganic phosphor particles should be of the type capable of severely milling and reducing materials to particles having sizes about 5 μm, or smaller, particularly below 1 μm, e.g., through mechanical action. Such mills are commercially available. The wet-mill can be a standard milling equipment, such as, for example, a Dyno mill type KDL, Drais TEX or a Netzsch mill type LME. Preferably, the LabStar from Netzsch, the model LMZ from Netzsch, the Drais DCP Superflow, Drais Advantis and the Model Dyno MultiLab from WAB are used. By using said ball mills very small final crystals of the inorganic phosphor, having, for example, particle sizes below 200 nm can be obtained. These mills are operated at power densities >0.5 kW per litre of grinding space and are more efficient in terms of milling efficiency. The construction of the mills and especially the inlet of the milling chamber as well as the agitator shaft of the mill can be formed by a standard material, but is preferably formed by ceramic materials, such as, for example, silicon carbide or nitride, in order to minimize abrasion and to reduce the impurities in the milled product.

Examples of mills which are especially suitable for the milling carried out in the method of the invention are stirred ball mills which are designed for batchwise and continuous operation, which have a cylindrical or hollow-cylindrical milling chamber in horizontal or vertical construction, and which can be operated with a specific power density of more than 0.5 kW, in particular more than 0.65 kW, per litre of milling space, and whose peripheral stirrer speed is more than 12 m/s. Mills suitable for this purpose are described, for example, in DE-C-3 716 587. For excellent results, it has been found that the specific power density should be 1.5 to at most 2.0 kJ·s⁻¹ per litre of grinding space and the peripheral speed of the agitator should then be from 5 to 12 m·s⁻¹, preferably from 6 to 11 m·s⁻¹. Higher peripheral speeds of up to about 15 m·s⁻¹ (perhaps even higher in the future) are possible with some special apparatus, but only if achievable at a specific power density of at most 2.0 kJ·s⁻¹ per litre of grinding space.

The wet-mill is previously filled to 60 to 95%, especially 80 to 95%, with ceramic grinding beads (0.1 mm<d<3 mm; specific density >3 g/cm³) and is operated at a tip speed going from 8 to 20 m/sec, preferably 10-15 m/sec. Examples of ceramic grinding beads are zirconium oxide beads (ZrO₂, ZrSiOₓ, or ZrAl₂Oₓ), yttrium stabilized zirconia beads, yttrium stabilized zirconium silicate, or zirconia core/shell type composite milling media described in U.S. Pat. No. 6,491,239 and U.S. Pat. No. 6,309,749. Examples of commercially available ceramic beads made of yttrium stabilised zirconia are SiLibeads® Type ZY or Type ZY Premium (from Sigmund Linder) and Zirmill® Zirpro® (from Saint Gobain), which are preferably used in the process according to the present invention. The zirconium oxide beads have a diameter of from 0.05 mm to 1 mm, especially 0.2 to 0.3 mm.

The inorganic phosphor is suspended in water. The mixture is stirred until a uniform suspension is formed. Stirring can be effected by using, for example, a propeller stirrer. The stirring time is usually 10 to 180 minutes. The suspension is than pumped in re-circulation mode through a bead mill for 0.5-12 hours. In order to keep the temperature between 5-95° C., preferably 15-55° C., during the milling, heat is removed by external cooling of the mill.

Depending on the effective milling temperature, water is added to the mill-base in order to keep the viscosity below 500 mPa s, preferably below 200 mPa s. Usually, 1 to 40% by weight, especially 5 to 25% by weight of inorganic phosphor and 99 to 60% by weight, especially 95 to 75% by weight solvent are used, based on inorganic phosphor and solvent.

The treatment period of the inorganic phosphor in the agitated media pearl mill is usually from 0.5 to 12 h.

At the end of the milling, crystals of the inorganic phosphor are obtained, the particle size of which depends on the milling time and milling parameters.

If necessary, the milling process might be performed under exclosure of oxygen under the atmosphere of a protective gas, such as nitrogen and argon to protect the Phosphors from oxidation during milling or firing. Oxidation of components of the phosphor may be prevented by addition of small amounts of a reducing agent, such as, for example, Na₂S₂O₄.

Inorganic phosphors having a particle size below 1 μm can be obtained by the process of the present invention, which surprisingly develop enough fluorescence for applications that were not possible till now with classical phosphors.

Depending on the application domain, the milled aqueous suspension can be:
used as a milled slurry in aqueous applications, such as, for example, aqueous inks;
isolated by filtration and kept under its wet form;
spray dried, and optionally fired later in an oven at 50-1300° C., and preferably 150-1000° C.;
isolated by filtration and dried in an oven by 50-1300° C., preferably 70-1000° C.; or
dried in an oven (with or without vacuum) by 50-1300° C., preferably 70-1000° C.

Firing of the milled phosphor can improve the luminescence of the phosphor.

Further processing may also be needed to insure that essentially all of the distribution of particles is below 1 micron or less. In such a case, the milled dispersion is processed to separate the submicron particles from the particles greater than one micron. This separation can be created by centrifuging the milled inorganic phosphor particles into a supernatant phase, which comprises the particles of the final product, and a settled phase which comprises the larger particles. The supernatant phase is then removed from the settled phase, e.g., by decanting. The supernatant is the dispersion of this invention. Conventional centrifuges can be used for this phase separation. In some instances, it may be preferable to centrifuge the supernatant two, three or more times to further remove large particles remaining after the initial centrifuge and to obtain a more uniform particle size distribution.

The crude inorganic phosphors, which are used as starting material in the process of the present invention, are commercially available, for examples, as phosphors for lamps. “Crude inorganic phosphors” means an inorganic phosphor as it is present after synthesis and firing. Reference is made, for example, to chapter 4.2 of “Inorganic Phosphors, Compositions, Preparations and Optical Properties” edited by CRC Press (Author: M. Yen; Marvin J. Weber). Especially suitable crude inorganic phosphors are magnesiumfluorogermanate:Mn(Mg₈Ge₂O₁₁F₂:Mn)); Yttriumvanadatephosphate:Eu (YPVO₄:Eu³); Bariummagnesiumaluminate:Eu,Mn (BaMgAl₁₀O₁₇:Eu, Mn).

In principle, any inorganic phosphor can be used in the process of the present invention. Examples of inorganic phosphors are given below

### I) Sulfides and Selenides

### a) Zinc and Cadmium Sulfides and Sulfoselenides

The raw materials for the production of sulfide phosphors are high-purity zinc and cadmium sulfides, which are precipitated from purified salt solutions by hydrogen sulfide or ammonium sulfide. The Zn_{1-y}Cd_{y}S (0≦y≦0.3) can be produced by co-precipitation from mixed zinc-cadmium salt solutions.

The most important activators for sulfide phosphors are copper and silver, followed by manganese, gold, rare earths, and zinc. The charge compensation of the host lattice is effected by coupled substitution with mono- or trivalent ions (e.g., Cl⁻ or Al³⁺).

The luminescent properties can be influenced by the nature of the activators and co-activators, their concentrations, and the firing conditions. In addition, specific substitution of zinc or sulphur in the host lattice by cadmium or selenium is possible, which also influences the luminescent properties.

Doping zinc sulfide with silver (silver activation) leads to the appearance of an intense emission band in the blue region of the spectrum at 440 nm, which has a short decay time.

The substitution of zinc by cadmium in the ZnS:Ag phosphor leads to a shift of the emission maximum from the blue over to the green, yellow, red to the IR spectral region.

Activation with copper causes an emission in zinc sulfide which consists of a blue (460 nm) and a green band (525 nm) in varying ratios, depending on the preparation.

Zinc sulfide forms a wide range of substitutionally mixed crystals with manganese sulfide. Manganese-activated zinc sulfide has an emission band in the yellow spectral region at 580 nm.

The activation of zinc sulfide with gold leads to luminescence in the yellow-green (550 nm) or blue (480 nm) spectral regions, depending on the preparation, whereas a blue-white luminescent phosphor is formed on activation with phosphorus.

The activators are added in the form of oxides, oxalates, carbonates, or other compounds which readily decompose at higher temperatures.

### b) Alkaline-Earth Sulfides and Sulfoselenides

Activated alkaline-earth metal sulfides have emission bands between the ultraviolet and near infrared. The alkaline-earth sulfides, such as MgS, or CaS, activated with rare earths, such as europium, cerium, or samarium, are of great importance:

CaS:Ce³⁺ is a green-emitting phosphor. On activation with 10⁻⁴ mol % cerium, the emission maximum occurs at 540 nm. Greater activator concentrations lead to a red shift; substitution of calcium by strontium, on the other hand, leads to a blue shift. MgS:Ce³⁺ (0.1%) has two emission bands in the green and red spectral regions at 525 and 590 nm; MgS:Sm³⁺ (0.1%) has three emission bands at 575 nm (green), 610 (red), and 660 nm (red).

Calcium or strontium sulfides, doubly activated with europium-samarium or cerium-samarium, can be stimulated by IR radiation. Emission occurs at europium or cerium and leads to orange-red (SrS:Eu²⁺, Sm³⁺) or green (CaS:Ce³⁺, Sm³⁺) luminescence.

### c) Oxysulfides

The main emission lines of Y₂O₂S:Eu³⁺ occur at 565 and 627 nm. The intensity of the long-wavelength emission increases with the europium concentration, whereby the colour of the emission shifts from orange to deep red. Terbium in Y₂O₂S has main emission bands in the blue (489 nm) and green spectral regions (545 and 587 nm), whose intensity ratio depends on the terbium concentration. At low doping levels, Y₂O₂S:Tb³⁺ luminesces blue-white, while at higher levels the colour tends towards green. Gd₂O₂S:Tb³⁺ exhibits green luminescence.

### II) Oxygen-Dominant Phosphors

### a) Borates:

Sr₃B₁₂O₂₀F₂:Eu²⁺.

### b) Aluminates:

Yttrium aluminate Y₃Al₅O₁₂:Ce³⁺ (YAG) is produced by precipitation of the hydroxides with NH₄OH from a solution of the nitrates and subsequent firing.

Cerium magnesium aluminate (CAT) Ce_{0.65}Tb_{0.35}MgAl₁₁O₁₉ is produced by coprecipitation of the metal hydroxides from a solution of the nitrates with NH₄OH and subsequent firing. A strongly reducing atmosphere is necessary to ensure that the rare earths are present as Ce³⁺ and Tb³⁺. Examples of further aluminate phosphors are BaMg₂Al₁₆O₂₇:Eu²⁺ and Y₂Al₃Ga₂O₁₂:Tb³⁺.

Long decay phosphors that are comprised of rare-earth activated divalent, boron-substituted aluminates are disclosed in U.S. Pat. No. 5,376,303. In particular, the long decay phosphors are comprised of MOₐ(Al_{1-b}B_{b})₂O₃:c R¹⁰³, wherein 0.5≦a≦10.0, 0.0001≦b≦0.5 and 0.0001≦c≦0.2, MO represents at least one divalent metal oxide selected from the group consisting of MgO, CaO, SrO and ZnO and R¹⁰³ represents Eu and at least one additional rare earth element. Preferably, R¹⁰³ represents Eu and at least one additional rare earth element selected from the group consisting of Pt, Nd, Dy and Tm.

### c) Silicates

ZnSiO₄:Mn is used as a green phosphor. Its production involves the precipitation of a [Zn(NH₃)₄](OH)₂ and MnCO₃ solution onto the porous SiO_{z} flakes, which are subsequently dried and fired.

Yttrium orthosilicate Y₂SiO₅:Ce³⁺ can be produced by treating an aqueous solution of (Y, Tb)(NO₃)₃ with the SiO_{z} flakes, heating and by subsequent reductive firing under N₂/H₂. An yttrium orthosilicate can be doped with Ce, Tb, and Mn.

### d) Germanates

Magnesium fluorogermanate, 3.5 MgO.0.5 MgF₂.GeO₂:Mn⁴⁺ is a brilliant red phosphor.

### e) Halophosphates and Phosphates

The halophosphates are doubly activated phosphors, in which Sb³⁺ and Mn²⁺ function as sensitizer and activator, giving rise to two corresponding maxima in the emission spectrum. The antimony acts equally as sensitizer and activator. The chemical composition can be expressed most clearly as 3 Ca₃(PO₄)₂.Ca(F, Cl)₂:Sb³⁺, Mn²⁺. Examples are (Sr, Mg)₃(PO₄)₂:Sn²⁺; LaPO₄:Ce³⁺, Tb³⁺; Zn₃(PO₄)₂:Mn²⁺; Cd₅Cl(PO₄)₂:Mn²⁺; Sr₃(PO₄)₂.SrCl₂:Eu²⁺; and Ba₂P₂O₇:Ti⁴⁺.

3 Sr₃(PO₄)₂.SrCl₂:Eu²⁺ can be excited by radiation from the entire UV range. The excitation maximum lies at 375 nm and the emission maximum at 447 nm. Upon successive substitution of Sr²⁺ by Ca²⁺ and Ba²⁺, the emission maximum shifts to 450 nm.

### f) Oxides:

The preparation of Y₂O₃:Eu³⁺ is generally carried out by precipitating mixed oxalates from purified solutions of yttrium and europium nitrates. Firing the dried oxalates is followed by crystallization firing.

Y₂O₃:Eu³⁺ shows an intense emission line at 611.5 nm in the red region. The luminescence of this red emission line increases with increasing Eu concentration up to ca. 10 mol %. Small traces of Tb can enhance the Eu fluorescence of Y₂O₃:Eu³⁺.

ZnO:Zn is a typical example of a self-activated phosphor.

### g) Arsenates:

Magnesium arsenate 6 MgO.As₂O₅:Mn⁴⁺ is a very brilliant red phosphor. Its production comprises the precipitation of magnesium and manganese with pyroarsenic acid using solutions of MgCl₂ and MnCl₂.

### h) Vanadates

Examples of vanadates activated with rare earths are YVO₄:Eu³⁺, YVO₄ with Tm, Tb, Ho, Er, Dy, Sm, or In; GdVO₄:Eu; LuVO₄:Eu. The incorporation of Bi³⁺ sensitizes the Eu³⁺ emission and results in a shift of the luminescence colour towards orange.

### i) Sulfates:

Photoluminescent sulfates are obtained by activation with ions that absorb short-wavelength radiation, for example, Ce³⁺. Alkali-metal and alkaline-earth sulfates with Ce³⁺ emit between 300 and 400 nm. On additional manganese activation, the energy absorbed by Ce³⁺ is transferred to manganese with a shift of the emission into the green to red region. Water-insoluble sulfates are precipitated together with the activators and fired below the melting point.

### j) Tungstates and Molybdates

Magnesium tungstate MgWO₄ and calcium tungstate CaWO₄ are the most important self-activated phosphors. Magnesium tungstate has a high quantum yield of 84% for the conversion of the 50-270 nm radiation into visible light. On additional activation with rare-earth ions their typical emission also occurs. One Example of a molybdate activated with Eu³⁺ is Eu₂(WO₄)₃.

### III) Halide Phosphors

Luminescent alkali-metal halides can be produced easily in high-purity and as large single crystals. Through the incorporation of foreign ions (e.g., Tl⁺, Ga⁺, In⁺) into the crystal lattice, further luminescence centers are formed. The emission spectra are characteristic for the individual foreign ions.

Some important alkali-metal halide phosphors are listed in Table below:

| Host Crystal | Activator |
|---|---|
| LiI | Eu |
| NaI | Tl |
| CsI | Tl |
| CsI | Na |
| LiF | Mg |
| LiF | Mg, Ti |
| LiF | Mg, Na |

Examples of halide phosphors are CaF₂:Mn; CaF₂:Dy, (Zn, Mg)F₂:Mn²⁺, KMg F₃:Mn²⁺, MgF₂:Mn²⁺, (Zn, Mg)F₂:Mn²⁺.

The oxyhalides of yttrium, lanthanum, and gadolinium are good host lattices for activation with other rare-earth ions such as terbium, cerium, and thulium, such as LaOCI:Tb³⁺ and LaOBr:Tb³⁺. The activator concentration (Tb, Tm) is 0.01-0.15 mol %. By co-activation, with ytterbium, the afterglow can be reduced. Partial substitution of lanthanum by gadolinium in LaOBr:Ce³⁺ leads to an increase in the quantum yield upon electron excitation and an increase in the quenching temperature.

With respect to the inorganic fluorescent substances a colorless inorganic phosphor prepared by calcining a composition comprising a crystal of an oxide, sulfide, silicate, phosphate, tungstate, or the like, of Ca, Ba, Mg, Zn, Cd or the like, as a principal component with a metallic element such as Mg, Ag, Cu, Sb, or Pb, or a rare-earth element such as lanthanoids, added therein as an activating agent, can be used.

An inorganic phosphor emitting a red light that may be used includes, for example, Ln₂O₃:Eu; Ln₂VO₄:Eu; Ln(V,P)O₄:EU; Ln₂(V,P,B)O₄; Eu; Ln₂VO₄:Eu; Ln₂(V,P)O₄:Eu; Ln₂(V,P,B)O₄:Eu; Y₂O₃:Eu; YVO₄:Eu; Y(V,P)O₄:EU; Y(V,P,B)O₄; Eu; YVO₄:Eu; Y(V,P)O₄:Eu; Y(V,P,B)O₄; Eu; Mg₄GeO_{5,5}F:Mn; SrMg(SiO₄)₂:Eu,Mn; CaSnO₄:Eu; Mg₄(Ge, Sn)O_{5,5}:Mn; Y₂O₃:Eu; Ln₂O₃:Eu (Ln=Lanthanide); Gd(Zn, Mg)B₅O₁₀:Ce,Mn; (Y, Eu)W₃O₁₂ and the like.

An inorganic phosphor emitting a green light that may be used includes, for example, ZnSiO₄:Mn; (Ce, Tb)MgAl₁₁O₁₉; (Ce, Tb,Mn)MgAl₁₁O₁₉; LaPO₄:Ce, Tb; Y₂SiO₅:Ce, Tb; MgGa₂O₄:Mn, (Ba(Eu)(Mg(Mn)Al₁₆O₂₇; Zn₂SiO₄:Tb; Y₂O₃:Eu; Al₂O₃:Tb; Y₃Al₅O₁₂:Tb; SrAl₂O₄:Eu; and the like.

An inorganic phosphor emitting a blue or yellow light that may be used includes, for example, Y₃Al₅O₁₂:Ce; Y₃(Al, Ga)₅O₁₂:Ce, both yellow emission; and Sr₃Ca₂(PO₄)₃Cl:Eu; (SrBaCa)₅(PO₄)₃Cl:Eu; CaWO₄; CaWO₄:Pb; Ba, MgAl₁₀O₁₇:Eu,Mn, BaMg₂Al₁₆O₂₇:Eu,Mn; Ba, MgAl₁₀O₁₇:Eu; BaMg₂Al₁₆O₂₇:Eu, all blue emission, and the like.

In another preferred embodiment of the present invention the inorganic phosphors are inorganic phosphorescent substances emitting a blue, green, or red light.

Inorganic phosphorescent substances emitting a blue, or green light are, for example, described in EP-A1-0622440.

The inorganic phosphorescent substances described therein comprise a matrix of formula MAl₂O₄ wherein M is calcium, strontium or barium, or a matrix of formula (M′xM″y)Al₂O₄ wherein x+y=1 and M′ and M″, which are different, are each a metal selected from calcium, barium, strontium and magnesium. The matrix comprises europium as activator. The matrix comprises, as co-activator, at least one element selected from lanthanum, cerium, praseodymium, neodymium, samarium, gadolinium, terbium, dysprosium, holmium, erbium, thulium, ytterbium, lutetium, manganese, tin and bismuth.

The matrix comprises europium in an amount of 0.001 to 10 mol % relative to the metal or metals in the matrix. The co-activator is comprised in an amount of 0.001 to 10 mol % relative to the metal or metals in the matrix.

SrAl₂O₄:Eu (emits yellow green light)

### SrAl₂O₄:Eu, Dy

### SrAl₂O₄:Eu, Nd

CaAl₂O₄:Eu (emits blue light)

### CaAl₂O₄:Eu, Nd

### CaAl₂O₄:Eu, Sm

### CaAl₂O₄:Eu, Dy

### CaAl₂O₄:Eu, Th

BaAl₂O₄:Eu, Nd (emits blue light, 480 nm)
BaAl₂O₄:Eu, Sm (emits green light, 500 nm)
Sr_{0.5}Ca_{0.5}Al₂O₄:Eu, Dy (emits green light, 500 nm)
SrₓCa₁₋ₓAl₂O₄:Eu, Dy
SrₓBa₁₋ₓAl₂O₄:Eu, Dy
SrₓMg₁₋ₓAl₂O₄:Eu, Dy

Another example of a green emitting inorganic phosphorescent substance is ZnS:Cu.

In one embodiment of the present invention phosphors on the basis of metal(III) vanadates, or vanadate/phosphate are less preferred.

In another embodiment of the present invention phosphors based on particles of a rare-earth (Ln) phosphate, said material having a P/Ln molar ratio greater than 1, are less preferred.

Said inorganic phosphorescent substances exhibit (intense) phosphorescence (after glow) during and after irradiation with ultra violet light, or visible rays having a wavelength of 200 to 450 nm at room temperature.

A list of suitable phosphorescent inorganic material is shown below:

| Formula | Emission Colour |
|---|---|
| CaO:Eu³⁺ | orange |
| CaO:Tb³⁺ | green |
| SrO:Pb²⁺ | violet |
| SrO:Eu³⁺ | orange |
| SrO:Tb³⁺ | green |
| BaO:Eu³⁺ | red |
| Y₂O₂S:Ti⁴⁺,Mg²⁺ | |
| (Y_{2-x-y}TixMg_{y})O₂S | yellow-orange |
| Y₂O₂S:Sm²⁺,Ti⁴⁺,Mg²⁺ | red |
| Y₂O₂S:Eu³⁺,Ti⁴⁺,Mg²⁺ | red |
| Y₂O₂S:Tm³⁺,Ti⁴⁺,Mg²⁺ | bluegreen |
| Y₂O₂S:Yb³⁺,Ti⁴⁺,Mg²⁺ | |
| Y₂O₂S:Eu³⁺,Ti⁴⁺ | red |
| Y₂O₂S | |
| Y₂O₂S:RE³⁺ (RE:Lu/Gd) | green |
| Y₂O₂S:Tb³⁺ | white: |
| ,Sr²⁺ and/or Zr⁴⁺ | blue and yellow- |
| | green |
| Y₂O₂S:Tm³⁺ | orange-yellow |
| Gd₂O₂S:Er³⁺,Ti⁴⁺ | |
| CaS:Eu²⁺,Ce³⁺ | |
| CaS:Eu²⁺,Sm³⁺ | |
| CaS:Eu²⁺,Tm³⁺ | red |
| CaS:Eu²⁺,Tm³⁺, Ce³⁺ | red |
| (Ca,Sr)S:Bi³⁺ | blue |
| CaGa₂S₄:Eu²⁺,Ho³⁺ | yellow |
| CaGa₂S₄:Eu²⁺,RE³⁺ | |
| (RE:Y/Ce/Pr/Gd/Tb/Ho) | |
| SrS:Eu²⁺, Y³⁺, Ce³⁺ | orange |
| ZnS:Cu | yellow-green |
| ZnS:Cu, Co | yellow-green |
| Zn₄O(BO₂)₆ | violet |
| CaAl₂B₂O₇:Eu²⁺,Nd³⁺ | blue |
| MgAl₂O₄:Ce³⁺ | green |
| CaAl₂O₄:Mn²⁺,Ce³⁺ | green |
| CaAl₂O₄:Eu²⁺,Nd³⁺ | blue |
| Ca_{1-x-y}Al₂O₄:Euₓ²⁺,Nd_{y}³⁺ | blue |
| (0 ≦ x ≦ 0.045; 0 ≦ y ≦ 0.0037) | |
| opt.: x = 0.00125; y = 0.0025 | |
| CaAl₂O₄:Eu²⁺,Nd³⁺ | blue |
| CaAl₂O₄:Eu²⁺,Nd³⁺,La³⁺ | blue-violet |
| CaAl₄O₇:Eu²⁺,Nd³⁺ | |
| Ca1 − xSrxAl₂O₄:Eu²⁺,Nd³⁺,La³⁺ | |
| SrAl₂O₄:Ce³⁺ | |
| SrAl₂O₄:Eu²⁺ | green |
| SrAl₂O₄:Eu²⁺,B³⁺ | green |
| SrAl₂O₄:Eu²⁺,Nd³⁺ | |
| SrAl₂O₄:Eu²⁺,Dy³⁺ | green |
| MAl₂O₄:Eu²⁺, Dy³⁺ M:Sr, (Ba/Ca) or M:Sr,Ba,Ca | f(m) |
| Sr₄Al₁₄O₂₅:Eu²⁺,RE³⁺ RE:Dy/Pr/Ho/Nd and/or Sm | |
| Sr₄Al₁₄O₂₅:Cr³⁺,Eu²⁺,Dy³⁺ | red-blue |
| Sr₅Al₂O₇S:Eu²⁺ | |
| Y₃Ga₅O₁₂:Cr³⁺ | |
| MgSiO₃:Mn²⁺,Eu²⁺,Dy³⁺ | red |
| SrSiO₃:Dy³⁺ | white: blue and |
| | yellow |
| CdSiO₃:In³⁺ | |
| CdSiO₃:Pb²⁺ | |
| CdSiO₃:Pr³⁺ | |
| CdSiO₃:Sm³⁺ | pink |
| CdSiO₃:RE³⁺ RE:Y/La/Gd/Lu | violet |
| CdSiO₃:RE³⁺ | f(re) |
| CdSiO₃:RE₁³⁺,RE₂³⁺ | f(re) |
| CdSiO₃:Mn²⁺,RE³⁺ RE:Y/La/Gd/Lu | orange |
| Ba₂SiO₄:Eu²⁺ | |
| Ba₃SiO₅:Eu²⁺ | |
| MO-M′O—SiO₂:Eu²⁺ | |
| M:Ca/Sr/Ba | |
| M′:Mg/Zn/Cd | blue-yellow f(m,s) |
| or | |
| MO-M′O—SiO₂:Eu²⁺,RE | |
| M:Ca/Sr/Ba, M′:Mg/Zn/Cd | blue-yellow f(m,s) |
| BaMg₂Si₂O₇:Mn²⁺,Eu²⁺,Dy³⁺ | red(mn) |
| BaMg₂Si₂O₇:Mn²⁺,Eu²⁺ (Ba-Defizit) | reddish |
| AMg₂Si₂O₇:Eu²⁺,Mn²⁺ | |
| A = Ba | violet |
| A = Sr | blue |
| A = Ca | yellow |
| Ca₂MgSi₂O₇:Eu²⁺,Dy³⁺ | |
| Sr_{0.5}Ca_{1.5}MgSi₂O₇:Eu²⁺,Dy³⁺ | green |
| (Ca,Sr)₂MgSi₂O₇:Eu²⁺,Dy³⁺ | |
| (Sr,Ca)MgSi₂O₇:Eu²⁺,Dy³⁺ | blue-green |
| Sr₂₋ₓCaₓMgSi₂O₇:Eu²⁺,Dy³⁺ | |
| x = 0 | blue |
| x = 0.5 | blue-green |
| x = 1 | green |
| x = 1.5 | yellow-green |
| x = 2 | yellow |
| x = 0 | |
| x = 0.8 | |
| x = 1.2 | |
| Sr₂MgSi₂O₇:Dy³⁺ | white: blue and |
| | yellow |
| Sr₂MgSi₂O₇:Eu²⁺,Nd³⁺ | blue |
| Sr₂MgSi₂O₇:Eu²⁺,Dy³⁺ | blue |
| Sr₂MgSi₂O₇:Eu²⁺,Dy³⁺ | blue |
| Sr₂₋ₓBaₓMgSi₂O₇:Eu²⁺,Dy³⁺/Nd³⁺,Cl⁻ (0 ≦ x ≦ 2) | |
| Sr₃MgSi₂O₈:Eu²⁺,Nd³⁺,Cl⁻ | |
| Ca₂Al₂SiO₇:Mn²⁺,Ce³⁺ | yellow |
| Ca_{0.5}Sr_{1.5}Al₂SiO₇:Ce³⁺,Tb³⁺ | white |
| Sr₃Al₁₀SiO₂₀:Eu²⁺,RE³⁺ | blue |
| (CaO —CaBr₂ —SiO₂):Eu²⁺ | green-yellow |
| NaGdGeO₄:Tb³⁺ | green |
| Zn₂GeO₄:Mn²⁺ | green |
| Cd₃Al₂Ge₃O₁₂:RE³⁺ RE:Pr/Tb/Dy | |
| Mg₂SnO₄:Mn²⁺ | green |
| Zn₃(PO₄)₂:Mn²⁺,M³⁺ M:Al,Ga | |
| Zn₃(PO₄)₂:Mn²⁺,Ga³⁺ | red |
| Zn₃(PO₄)₂:Mn_{0.052}²⁺,Ga³⁺ | red |
| Zn₃(PO₄)₂:Mn²⁺,Zr⁴⁺ | red(mn), blue(zr) |
| Zn₃(PO₄)₂:Mn²⁺,Sm³⁺ | red |
| Ba₂TiP₂O₉ | white |
| CaTiO₃:Pr³⁺ | red |
| Ca_{0.8}Zn_{0.2}TiO₃:Pr³⁺ | red |
| Ca₂Zn₄Ti₁₅O₃₆:Pr³⁺ | red |
| Y_{1-y}NbO_{2.5 + 1.5y}:Bi³⁺ (non-stoichiometric) | violet |

In a preferred embodiment of the present invention the inorganic phosphor is not a phosphor which satisfies with the general formula A₃Cr₅₋ₓAlₓO₁₂, where A stands for an element selected from the group consisting of scandium, yttrium, the lanthanides and the actinides, and the index x fulfils the condition 0<x<4.99.

In a preferred embodiment of the present invention the inorganic phosphor is not a phosphor which is represented by the general formula (Ca₂₋ₓ, Srₓ)P₂O₇:Pr where 0≦x≦2.

In a preferred embodiment of the present invention the inorganic phosphor is not a rare earth borate which is embodied in the form of a liquid phase suspension of substantially monocrystalline particles whose mean size ranges from 100 to 400 nm.

The inorganic phosphorescent substances exhibit intense phosphorescence during and after irradiation with visible, or ultra violet light.

In practice either visible light, long-wavelength UV (365 nm, or 395 nm) or short-wavelength UV (254 nm) is generally used to induce phosphorescence. The phosphorescence represents the radiative decay of a triplet excited state to the singlet ground state; this transition is forbidden and the triplet state has a relatively long lifetime.

At the end of the milling, depending on the milling time and parameters, crystals from 10 nm to 1μ can be formed. Surprisingly, the latter are still developing enough fluorescence for applications that were not possible till now with classical phosphors having particle sizes of 4 to 12 μm.

Inkjet printing, (security) printing, plastics (more resistant fibres), thin layer coating/printing, thin phosphors for lamps are now possible.

Accordingly, the inorganic phosphors can be used in paints, lacquers, printing inks, powder coatings, paper coatings, plastics, cosmetics, inks, glazes for ceramics and glasses, decorative applications for foods and drugs and security-enhancing features and the present invention relates also to paints, lacquers, printing inks, powder coatings, paper coatings, plastics, cosmetics, inks, glazes for ceramics and glasses, comprising the inorganic phosphor according to the present invention, including a product for forgery prevention comprising the inorganic phosphor according to the present invention.

The inorganic phosphors can be provided with an additional stabilising protective layer, the so-called post-coating, which simultaneously effects optimum adaptation to the binder system. The protective layer comprises one, or more metal oxides and/or an organic chemical surface modification. The metal oxide/hydroxide of the protective layer is preferably selected from oxides/hydroxides of silicon (silicon oxide, silicon oxide hydrate), aluminum, zirconium, magnesium, calcium, iron(III), yttrium, cerium, zinc, bor and combinations thereof.

In a preferred embodiment of the present invention the metal oxide/hydroxide is an oxide/hydroxide of silicium, aluminum (aluminum oxide, aluminum oxide hydrate), zirconium ((hydrated) zirconium dioxide), or a mixture thereof.

The organic chemical surface modification is composed preferably of one or more organofunctional silanes, aluminates, zirconates and/or titanates. With very great preference the organic chemical surface modification is composed of one or more organofunctional silanes applied to the metal oxide(s) surface.

Various features and aspects of the present invention are illustrated further in the examples that follow. While these examples are presented to show one skilled in the art how to operate within the scope of this invention, they are not to serve as a limitation on the scope of the invention where such scope is only defined in the claims. Unless otherwise indicated in the following examples and elsewhere in the specification and claims, all parts and percentages are by weight, temperatures are in degrees centigrade and pressures are at or near atmospheric.

### EXAMPLES

The luminescence (fluorescence, or phosphorescence) is measured by exciting the inorganic phosphor in powder form by using an UVC emitting lamp and measuring the emission by a spectral radiometer (luminance in cd/m²).

### Example 1

200 g of crude Y(PV)O₄:Eu phosphor (average particle size=8.0 μm; D₅₀<7 μm) is flushed into a storage vessel and slurred in water (total weight of the suspension: 1000 g). The slurry is then passed, via a cylindrical wet mill (Netzsch LabStar), filled to about 90% of its volume with mixed zirconium oxide grinding elements from 0.3 to 0.4 mm in diameter, at a radial speed of 12 m·s⁻¹. The mixture is passed in recirculation mode through the mill and back to the storage vessel for 4 hours. The product is then filtered and washed and dried in customary manner. A Y(PV)O₄:Eu phosphor having an average particle size=0.06 μm; a D₅₀<0.05 μm, D₉₀<0.07 μm and a narrow particle size distribution (distribution coefficient (D₁₀+D₉₀)/D₅₀<1.2) is obtained. The Y(PV)O₄:Eu phosphor obtained after wet milling shows 60% of the luminescence of the initial crude Y(PV)O₄:Eu phosphor.

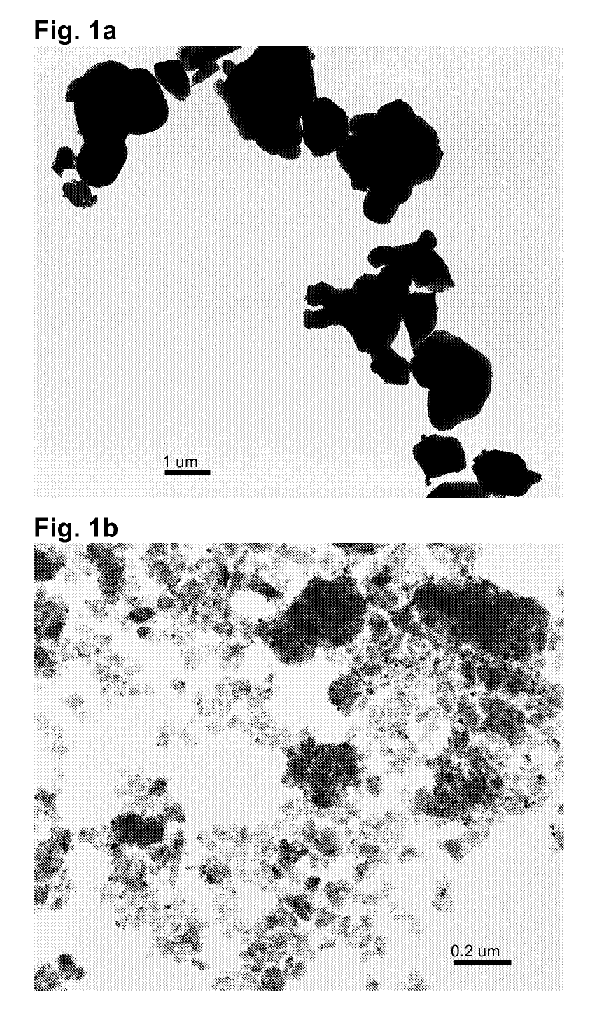

FIG. 1a is a transmission electron micrograph (TEM) of the crude Y(PV)O₄:Eu phosphor.

FIG. 1b is a transmission electron micrograph (TEM) of the Y(PV)O₄:Eu phosphor obtained after wet milling.

### Example 2

200 g of a crude Mg₈Ge₂O₁₁F₂:Mn phosphor (average particle size=7 μm; D₅₀<6.5 μm) is flushed into a storage vessel and slurred in water (total weight of the suspension: 1000 g). The slurry is then passed, via a cylindrical wet mill (Netzsch LabStar®), filled to about 90% of its volume with mixed zirconium oxide grinding elements from 0.3 to 0.4 mm in diameter, at a radial speed of 12 m·s⁻¹. The mixture is passed in recirculation mode through the mill and back to the storage vessel for 4 hours. The product is then filtered and washed and dried in a vacuum oven at 70° C. After firing in an oven at 850° C. a Mg₈Ge₂O₁₁F₂:Mn phosphor having an average particle size=0.07 μm; a D₅₀<0.08 μm, D₉₀<0.10 μm and a narrow particle size distribution (distribution coefficient (D₁₀+D₉₀)/D₅₀<1.2) is obtained. The Mg₈Ge₂O₁₁F₂:Mn:Eu phosphor obtained after wet milling and firing shows 91% of the luminescence of the initial crude Mg₈Ge₂O₁₁F₂:Mn:Eu phosphor.

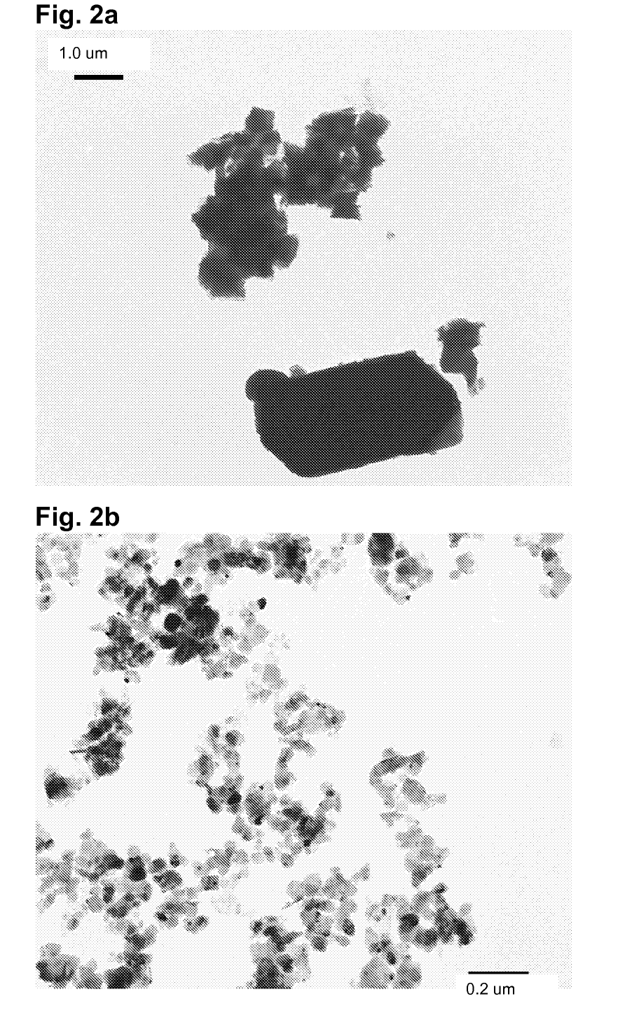

FIG. 2a is a transmission electron micrograph (TEM) of the crude Mg₈Ge₂O₁₁F₂:Mn phosphor.

FIG. 2b is a transmission electron micrograph (TEM) of the Mg₈Ge₂O₁₁F₂:Mn phosphor obtained after wet milling.

### Example 3

200 g of a crude blue BaMgAl₁₀O₁₇:Eu,Mn phosphor (Eu>Mn, average particle size=6.4 μm; D₅₀<6 μm) is flushed into a storage vessel and slurred in water (total weight of the suspension: 1000 g). The slurry is then passed, via a cylindrical wet mill (Netzsch LabStar®), filled to about 90% of its volume with mixed zirconium oxide grinding elements from 0.3 to 0.4 mm in diameter, at a radial speed of 12 m·s⁻¹. The mixture is passed in recirculation mode through the mill and back to the storage vessel for 4 hours. The product is then filtered and washed and dried in a vacuum oven at 70° C. A BaMgAl₁₀O₁₇:Eu,Mn phosphor having an average particle size=0.09 μm; a D₅₀<0.1 μm, D₉₀<0.12 μm and a narrow particle size distribution (distribution coefficient (D₁₀+D₉₀)/D₅₀<1.2) is obtained. The BaMgAl₁₀O₁₇:Eu,Mn phosphor obtained after wet milling shows 50% of the luminescence of the initial crude BaMgAl₁₀O₁₇:Eu,Mn phosphor.

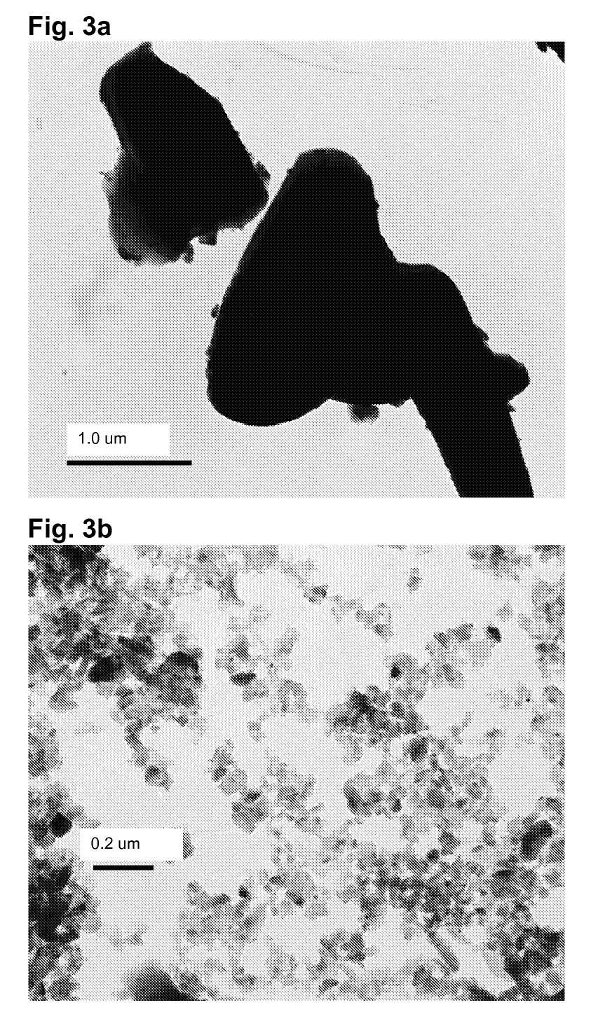

FIG. 3a is a transmission electron micrograph (TEM) of the crude BaMgAl₁₀O₁₇:Eu,Mn phosphor.

FIG. 3b is a transmission electron micrograph (TEM) of the BaMgAl₁₀O₁₇:Eu,Mn phosphor obtained after wet milling.

### Example 4

200 g of crude Y(PV)O₄:Eu phosphor (average particle size=8.0 μm; D₅₀<7 μm) is flushed into a storage vessel and slurred in water (total weight of the suspension: 1000 g). The slurry is then passed, via a cylindrical wet mill (Netzsch LabStar), filled to about 90% of its volume with mixed zirconium oxide grinding elements from 0.3 to 0.4 mm in diameter, at a radial speed of 12 m·s⁻¹. The mixture is passed in recirculation mode through the mill and back to the storage vessel for 1 hour. The product is then filtered and washed and dried in customary manner. A Y(PV)O₄:Eu phosphor having an average particle size=0.13 μm; a D₅₀<0.15 μm, D₉₀<0.18 μm and a narrow particle size distribution (distribution coefficient (D₁₀+D₉₀)/D₅₀<1.2) is obtained. The Y(PV)O₄:Eu phosphor obtained after wet milling shows 70% of the luminescence of the initial crude Y(PV)O₄:Eu phosphor.

1. An inorganic phosphor, obtained by wet milling, wherein the mill is operated at power densities >0.5 kW per litre of grinding space and the luminescence (fluorescence, or phosphorescence) intensity of the wet milled inorganic phosphor is at least about 50% of the luminescence intensity of the inorganic phosphor, which is used as starting material in the milling process.

2. The inorganic phosphor according to claim 1, wherein the inorganic phosphor has a particle size distribution of D90≦5 μm.

3. The inorganic phosphor according to claim 1, wherein at least about 90 weight percent of the particles are not larger than twice the average particle size.

4. The inorganic phosphor according to claim 3, wherein at least about 90 weight percent of the particles are not larger than about 1.5 times the average particle size.

5. The inorganic phosphor according to claim 1, wherein the phosphor particles are characterized by a distribution coefficient (D₁₀+D₉₀)/D₅₀<1.2.

6. The inorganic phosphor according to claim 1, wherein the average particle size and/or the D₅₀ of the inorganic phosphors is below 0.4 μm.

7. The inorganic phosphor according to claim 1, wherein the inorganic phosphor is Ln₂O₃:Eu; Ln₂VO₄:Eu; Ln(V,P)O₄:EU; Ln₂(V,P,B)O₄; Eu; Ln₂VO₄:Eu; Ln₂(V,P)O₄:Eu; Ln₂(V,P,B)O₄:Eu; Y₂O₃:Eu; YVO₄:Eu; Y(V,P)O₄:EU; Y(V,P,B)O₄; Eu; YVO₄:Eu; Y(V,P)O₄:Eu; Y(V,P,B)O₄; Eu; Mg₄GeO_{5,5}F:Mn; SrMg(SiO₄)₂:Eu,Mn; CaSnO₄:Eu; Mg₄(Ge, Sn)O_{5,5}:Mn; Y₂O₃:Eu; Ln₂O₃; Eu (Ln=Lanthanide), Gd(Zn, Mg)B₅O₁₀:Ce,Mn; (Y, Eu)W₃O₁₂; ZnSiO₄:Mn; (Ce, Tb)MgAl₁₁O₁₉; (Ce, Tb,Mn)MgAl₁₁O₁₉; LaPO₄:Ce, Tb; Y₂SiO₅:Ce, Tb; MgGa₂O₄:Mn, (Ba(EU)(Mg(Mn)Al₁₆O₂₇; Zn₂SiO₄:Tb; Y₂O₃; Al₂O₃:Tb; Y₃Al₅O₁₂:Tb; SrAl₂O₄:Eu; Y₃Al₅O₁₂:Ce; Y₃(Al, Ga)₅O₁₂:Ce; Sr₃Ca₂(PO₄)₃Cl:Eu; (SrBaCa)₅(PO₄)₃Cl:Eu; CaWO₄; CaWO₄:Pb; Ba, MgAl₁₀O₁₇:Eu,Mn, BaMg₂Al₁₆O₂₇:Eu,Mn; Ba, MgAl₁₀O₁₇:Eu; and BaMg₂Al₁₆O₂₇:Eu, or CaO:Eu³⁺, CaO:Tb³⁺, SrO:Pb²⁺, SrO:Eu³⁺, SrO:Tb³⁺, BaO:Eu³⁺, Y₂O₂S:Ti⁴⁺, Mg²⁺, (Y_{2-x-y}TixMg_{y})O₂S, Y₂O₂S:Sm²⁺, Ti⁴⁺, Mg²⁺, Y₂O₂S:Eu³⁺, Ti⁴⁺, Mg²⁺, Y₂O₂S:Tm³⁺, Ti⁴⁺, Mg²⁺, Y₂O₂S:Yb³⁺, Ti⁴⁺, Mg²⁺, Y₂O₂S:Eu³⁺, Ti⁴⁺, Y₂O₂S, Y₂O₂S:RE³⁺, (RE:Lu/Gd), Y₂O₂S:Tb³⁺, Sr²⁺ and/or Zr⁴⁺, Y₂O₂S:Tm³⁺, Gd₂O₂S:Er³⁺, Ti⁴⁺, CaS:Eu²⁺, Ce³⁴, CaS:Eu²⁺, Sm³⁺, CaS:Eu²⁺, Tm³⁺, CaS:Eu²⁺, Tm³⁺, Ce³⁺, (Ca, Sr)S:Bi³⁺, CaGa₂S₄:Eu²⁺, Ho³⁺, CaGa₂S₄:Eu²⁺, RE³⁺, (RE:Y/Ce/Pr/Gd/Tb/Ho), SrS:Eu²⁺, Y³⁺, Ce³⁺, ZnS:Cu, ZnS:Cu, Co, Zn₄O(BO₂)₆, CaAl₂B₂O₇:Eu²⁺, Nd³⁺, MgAl₂O₄:Ce³⁺, CaAl₂O₄:Mn²⁺, Ce³⁺, CaAl₂O₄:Eu²⁺, Nd³⁺, Ca_{1-x-y}Al₂O₄:Euₓ²⁺, Nd_{y}³⁺, (0≦x≦0,045; 0≦y≦0,0037), opt.:x=0,00125; y=0,0025, CaAl₂O₄:Eu²⁺, Nd³⁺, CaAl₂O₄:Eu²⁺, Nd³⁺, La³⁺, CaAl₄O₄:Eu²⁺, Nd³⁺, Ca1-x=SrxAl₂O₄:Eu²⁺, Nd³⁺, La³⁺, SrAl₂O₄:Ce³⁺, SrAl₂O₄:Eu²⁺, SrAl₂O₄:Eu²⁺, B³⁺, SrAl₂O₄:Eu²⁺, Nd³⁺, SrAl₂O₄:Eu²⁺, Dy³⁺, MAl₂O₄:Eu²⁺, Dy³⁺M:Sr, (Ba/Ca) or M:Sr, Ba, Ca, Sr₄Al₁₄O₂₅:Eu²⁺, RE³⁺RE:Dy/Pr/Ho/Nd and/or Sm, Sr₄Al₁₄O₂₅:Cr³⁺, Eu²⁺, Dy³⁺, Sr₅Al₂O₇S:Eu²⁺, Y₃Ga₅O₁₂:Cr³⁺, MgSiO₃:Mn²⁺, Eu²⁺, Dy³⁺, SrSiO₃:Dy³⁺, CdSiO₃:In³⁺, CdSiO₃:Pb²⁺, CdSiO₃:Pr³⁺, CdSiO₃:Sm³⁺, CdSiO₃:RE³⁺RE:Y/La/Gd/Lu, CdSiO₃:RE³⁺, CdSiO₃:RE₁³⁺, RE₂³⁺, CdSiO₃:Mn²⁺, RE³⁺RE:Y/La/Gd/Lu, Ba₂SiO₄:Eu²⁺, Ba₃SiO₅:Eu²⁺, MO-M′O—SiO₂:Eu²⁺, M:Ca/Sr/Ba, M′:Mg/Zn/Cd, or, MO-M′O—SiO₂:Eu²⁺, RE, M:Ca/Sr/Ba, M′:Mg/Zn/Cd, BaMg₂Si₂O₇:Mn²⁺, Eu²⁺, Dy³⁺, BaMg₂Si₂O₇:Mn²⁺, Eu²⁺ (Ba-Defizit), AMg₂Si₂O₇:Eu^{2'},Mn²⁺, A=Ba, A=Sr, A=Ca, Ca₂MgSi₂O₇:Eu²⁺, Dy³⁺, Sr_{0,5}Ca_{1,5}MgSi₂O₇:Eu²⁺, Dy³⁺, (Ca, Sr)₂MgSi₂O₇:Eu²⁺, Dy³⁺, (Sr, Ca)MgSi₂O₇:Eu²⁺, Dy³⁺, Sr₂₋ₓCaₓMgSi₂O₇:Eu²⁺, Dy³⁺, x=0, x=0,5, x=1, x=1.5, x=2, x=0, x=0,8, x=1,2, Sr₂MgSi₂O₇:Dy³⁺, Sr₂MgSi₂O₇:Eu²⁺, Nd³⁺, Sr₂MgSi₂O₇:Eu²⁺, Dy³⁺, Sr₂MgSi₂O₇:Eu²⁺, Dy³⁺, Sr₂₋ₓBaₓMgSi₂O₇:Eu²⁺, Dy³⁺/Nd³⁺, Cl⁻ (0≦x≦2), Sr₃MgSi₂O₈:Eu²⁺, Nd³⁺, Cl⁻, Ca₂Al₂SiO₇:Mn²⁺, Ce³⁺, Ca_{0,5}Sr_{1,5}Al₂SiO₇:Ce³⁺, Tb³⁺, Sr₃Al₁₀SiO₂₀:Eu²⁺, RE³⁺, (CaO—CaBr₂—SiO₂):Eu²⁺, NaGdGeO₄:Tb³⁺, Zn₂GeO₄:Mn²⁺, Cd₃Al₂Ge₃O₁₂:RE³⁺RE:Pr/Tb/Dy, Mg₂SnO₄:Mn²⁺, Zn₃(PO₄)₂:Mn²⁺, M³⁺M:Al, Ga, Zn₃(PO₄)₂:Mn²⁺, Ga³⁺, Zn₃(PO₄)₂:Mn_{0,052}²⁺, Ga³⁺, Zn₃(PO₄)₂:Mn²⁺, Zr⁴⁺, Zn₃(PO₄)₂:Mn²⁺, Sm³⁺, Ba₂TiP₂O₉, CaTiO₃:Pr³⁺, Ca_{0,8}Zn_{0,2}TiO₃:Pr³⁺, Ca₂Zn₄Ti₁₅O₃₆:Pr³⁺, or Y_{1-y}NbO_{2,5+1,5y}:Bi³⁺ (non-stoichiometric).

8. A method for the preparation of the inorganic phosphor of claim 1, comprising
a) forming a suspension of the crude inorganic phosphor in a liquid and optionally a neutral, polar liquid; and
b) wet milling said mixture, wherein the mill is operated at power densities >0.5 kW per litre of grinding space, and
c) optionally firing the milled phosphor.

9. The method according to claim 8, wherein the neutral, polar liquid is acetamide, formamide, methylacetamide, methylformamide, caprolactam, valerolactam, 1,1,2,2-tetramethylurea, dimethyl sulfoxide, sulfolane, nitromethane, nitrobenzene, acetonitrile, methanol, ethylene carbonate, dimethylacetamide, dimethylformamide and N-methylpyrrolidone, preferably dimethyl sulfoxide, dimethylformamide or N-methylpyrrolidone or is a mixture of a plurality of neutral liquids of same overall polarity.

10. The method according to claim 8, wherein the amount of neutral, polar liquid is from 1 to 30% by weight, based on the total amount of liquid and water.

11. The method according to claim 8, wherein the inorganic phosphor has a particle size distribution of D90≦5 μm.

12. A product for forgery prevention comprising the inorganic phosphor according to claims 1.

13. (canceled)

14. Paints, lacquers, printing inks, powder coatings, paper coatings, plastics, cosmetics, inks, glazes for ceramics and glasses, comprising the inorganic phosphor according to claim 1.
